(19) United States
(12) Patent Application Publication
(10) Pub. No.: US 2014/0234223 A1
(43) Pub. Date: Aug. 21, 2014

(54) NANOEMULSIONS AND USE THEREOF AS CONTRAST AGENTS

(71) Applicant: GUERBET, Villepinte (FR)

(72) Inventors: Marc Port, Deuil La Barre (FR); Caroline Robic, Nogent Sur Marne (FR); Jean-Michel Fabicki, Aulnay Sous Bois (FR)

(73) Assignee: GUERBET, Villepinte (FR)

(21) Appl. No.: 14/347,543

(22) PCT Filed: Sep. 20, 2012
(86) PCT No.: PCT/EP2012/068491

§ 371 Date: Mar. 26, 2014

(30) Foreign Application Priority Data
Sep. 26, 2011 (FR) 1158563

(51) Int. Cl. A61K 49/18; A61K 49/14; A61K 49/12; A61K 49/10

(52) U.S. Cl. CPC A61K 49/1806; A61K 49/10; A61K 49/14; A61K 49/126
USPC 424/9.36; 424/9.37

(57) ABSTRACT

The invention relates to an oil-in-water nanoemulsion for MRI, including:
an aqueous phase,
a fluorinated phase including at least one fluorinated oil,
a surfactant at the interface between the aqueous and fluorinated phases, the surfactant comprising:
   at least one amphiphilic targeting ligand,
   at least one amphiphilic lipid, and
   at least one diblock or triblock fluorophilic compound,
      as well as to the use thereof as a contrast agent.

The invention relates to novel optimized systems of nanoemulsion type and to the use thereof as contrast agents, in particular in MRI.

In the diagnostic imaging field, a great deal of research has related to lipid nanosystems of emulsion type. Typically, the emulsions used are in the form of vesicles prepared using lipid constituents (oil in particular) and surfactants acting as an interface between the aqueous phase and the lipid core of the nanoparticle. Oil-in-water lipid emulsions incorporate a lipophilic oily phase, forming lipid droplets in aqueous solution.

A first emulsion category described in particular in WO 03/062198 or U.S. Pat. No. 6,676,963 is that of fluorinated nanoemulsions comprising, integrated inside the lipid vesicles, fluorinated compounds comprising fluorine F19 atoms used for MRI magnetic resonance imaging. Indeed, fluorine has in particular the advantage, with respect to proton MRI, of being virtually absent from biological systems in the free state, thereby allowing it to be recognized as an excellent quantitative probe in the form of fluorine F19. The lipid core is made up of a fluorinated oil, and is surrounded by a lipid layer formed by a surfactant (lecithin, for example).

These fluorinated emulsions may also comprise a very high level of complexes of paramagnetic metals, in particular of lanthanides, for combining fluorine 19F and proton 1H MRI. Fluorinated emulsions for MRI incorporating chelates capable of complexing lanthanides, in particular gadolinium, are thus known. The chelates used are in particular derivatives of DTPA, DOTA, DO3A or HPDO3A and other chelates widely described in the prior art. These hydrophilic chelates are made lipophilic by grafting thereto a lipophilic region such as a phospholipid, which makes it possible to integrate them into the lipid membrane formed by the lipid surfactant of the composition. Several thousand (approximately 5000 to 100 000) of these complexes are integrated into the lipid membrane of the vesicles, thereby making it possible to obtain a high relaxivity (MRI signal) for detection of the physiological region studied and to modify the relaxation time of 19F. The hydrophilic part (the hydrophilic part represented by the chelate to which a lipophilic group is attached so as to take the amphiphilic chelate) is located at the external surface of the nanodroplets, in contact with the aqueous phase of the nanodroplet solution.

Oil-in-water nonfluorinated emulsions, comprising lanthanide chelates for solely proton MRI, are also known.

In addition, in order to obtain a signal specific for pathological regions, for example associated with an overexpression of a marker for these regions (receptors, for example), targeting molecules (or targeting ligands, peptide for example having an affinity for the receptor), have been grafted onto the nanodroplets of these fluorinated emulsions. WO 03/062198 describes in particular the use of peptidomimetic compounds for targeting integrins overexpressed in tumor regions. For incorporation into the lipid membrane, the targeting ligands (which are often hydrophilic) are made amphiphilic by combining them with lipophilic chains.

However, despite promising advances, the vectorized fluorinated contrast agents described have not yet completely demonstrated their clinical efficacy, and pose difficulties in terms of stability over time.

Compared with the known fluorinated emulsions, it is sought:
to improve the stability of the targeting ligands in these emulsions, all the more so since the industrial cost price of nanoemulsions is approximately at least 80% to 90% represented by the targeting ligands which are very expensive,
to increase the amount and the efficiency of incorporation of the targeting ligands, and the affinity of the emulsions for the biological target,
to achieve stability over time of at least one year, and preferably from 2 to 3 years.

It is recalled that the optimization of the constituents is complex: nature and content of the oil, of the surfactants, of the targeting ligands. For example, a surfactant content that is too high is reflected by:
the formation in the composition, in addition to the nanodroplets, of micelles, the removal of which would require, for an industrial-scale production, hundreds of tonnes of product, and complex and expensive separation and purification steps, hence a drop in the industrial yield,
the difficulty or even impossibility of incorporating into the nanoparticles an appropriate amount of biological targeting ligands, the cost of which is very high. Indeed, amphiphilic targeting ligands are often poorer surfactants than the surfactants used to stabilize the interface. As a result, the surface of the droplets is mainly occupied by the layer of surfactant amphiphilic lipids, and the targeting ligands have trouble integrating into this layer.

Other categories of emulsions for medical imaging exist, in particular nanoemulsions for fluorescence imaging, which do not comprise fluorinated compounds, and which use metal oxide nanocrystals. Document WO 2010/018222 describes such nanoemulsions comprising:
an aqueous solution,
a dispersed phase (oil) forming lipid nanodroplets in the aqueous solution, the nanodroplets incorporating nanocrystals of metal oxides having luminescence properties for fluorescence imaging,
a surfactant (for example phospholipids) and cosurfactants for stabilizing the nanodroplets.

However, fluorescence imaging is not very suitable for several major diagnostic indications, in particular some imaging of vascular and/or tumor territories.

In the light of the complex prior art, the difficulty in obtaining very effective nanoemulsions, in particular vectorized fluorinated nanoemulsions for fluorine MRI, which meet the constraints of industrialization and are clinically effective in a broad spectrum of indications, can be seen.

The applicant has succeeded in obtaining fluorinated nanoemulsions in the form of vectorized droplets:
which are sufficiently colloidally stable to be produced and stored for a long period of time, in particular without any problem of coalescence of the lipid droplets with one another,
which are sufficiently stable in vivo so as not to be degraded,
which are suitable from the pharmacokinetic point of view,
which are sufficiently effective in terms of the signal for clinical imaging in a patient,
which are capable of incorporating ligands for targeting pathological regions at the surface of the nanodroplets, in an appropriate amount and without any impairing loss of affinity with their biological target.

For this, the applicant has incorporated, into the prior nanoemulsions, fluorophilic dispersing agents, denoted diblock or triblock fluorophilic compounds.

To this effect, according to a first aspect, the invention relates to an oil-in-water nanoemulsion composition comprising:
an aqueous phase,
a fluorinated phase comprising at least one fluorinated oil,
a surfactant at the interface between the aqueous and fluorinated phases, the surfactant comprising:
   at least one amphiphilic targeting ligand,
   at least one amphiphilic lipid, and
   at least one diblock or triblock fluorophilic compound.

The term “nanoemulsion” is intended to mean that the droplet size is between 1 and 1000 nm. The droplet size is typically from 50 to 400 nm, advantageously 100 to 350 nm, especially 150 to 300 nm, and in particular 200 to 250 nm.

Surfactant

In the interests of simplification, it is indicated that the nanoemulsion comprises a surfactant. It is clear for those skilled in the art that this is a surfactant which forms a layer between the oily phase and the aqueous phase, and which is also denoted “total surfactants” in the application. As detailed later, the surfactant (total surfactants) comprises, on the one hand, nonfluorinated amphiphilic compounds and, on the other hand, fluorinated amphiphilic compounds, in particular the diblock or triblock fluorophilic compound.

Those skilled in the art understand that the surfactant at the fluorinated oil/aqueous phase interface corresponds to all the surfactants used, i.e. as explained in detail in the application: amphiphilic lipids, diblock or triblock fluorophilic compounds, amphiphilic targeting ligands and, optionally, in addition, amphiphilic paramagnetic metal chelates which may or may not be present depending on the embodiments, and, where appropriate, other compounds such as pegylated lipids (lipids coupled to PEGs). By virtue of their amphiphilic structure, the amphiphilic targeting ligands act as a surfactant, it being specified that the amount thereof is generally low compared with the other amphiphilic compounds used.

The expression “total surfactant” is intended to mean all of the surfactants in the composition.

Diblock or Triblock Fluorophilic Compound

The diblock or triblock fluorophilic compound is preferably written in the form FₙHₘ described in detail later.

Such diblock or triblock fluorophilic compounds are known in particular from document U.S. Pat. No. 5,733,526, but they are used therein to stabilize lipid systems of a certain type (oily micelles or droplets) incorporated into a nonaqueous system of another type (fluorinated oil), and not, as in the case in the present invention, to stabilize lipid systems in an aqueous phase. More specifically, document U.S. Pat. No. 5,733,526 describes in particular in its examples:
micelles with targeting ligands (the micelles not delimiting an aqueous internal compartment), incorporated into a fluorinated oil (PFOB), the diblocks or triblocks being directly the constituents of the micelles;
a carbon-based oil, incorporated into a fluorinated oil (PFOB), diblocks or triblocks being used to stabilize this interface.

Thus, in U.S. Pat. No. 5,733,526, the diblock fluorophilic compounds are located at the interface of a fluorinated oil and a nonfluorinated oil (hydrocarbon-based oil), whereas, in the present application, the diblock fluorophilic compounds are located at the interface between the fluorinated oil and the aqueous phase. Preferably, the nanoemulsion according to the invention is free of hydrocarbon-based oil.

Unexpectedly, the applicant has succeeded in demonstrating that its novel systems make it possible not only to obtain stable nanoemulsions, but also that the affinity of the oil-in-water nanoemulsions synthesized, for the biological target, is significantly improved.

The diblock fluorophilic compounds used for the nanoemulsions according to the invention advantageously have the general formula:

R_{F}-L-R_{H}(—Z)_{z}

in which:
1) R_{F} is a fluorinated or perfluorinated group (which optionally comprises side chains and/or rings and/or heteroatoms, in particular halogens);
2) R_{H} is a hydrocarbon-based group (which optionally comprises side chains and/or rings and/or heteroatoms, in particular halogens, and/or multiple bonds (for example —(CH₂)ₙ—, —C₆H₄(CH₂)₄—, —(CH₂)ₚO(CH₂)_{q}—, —(CH₂)₂CH═CH(CH₂)₅—; n, p and q being integers ranging from 1 to 50, in particular from 1 to 20, preferably from 2 to 10);
3) L is a linker group and may comprise in particular one of the following groups: single bond, —CH₂—, —CH═CH—, —O—, —S—, —PO₄—, CONH;
4) Z is H or a group which is more polar or polarizable than the R_{H} groups (for example an alcohol, a halogen, or an —O—R′_{H} group where R′_{H} is
   a hydrocarbon-based group, in particular an alkyl or a —(CH₂)ₘCH═CH₂ group with m being an integer ranging from 1 to 16, or
   a —P(O)[N(CH₂CH₂)₂O]₂) group;
5) z represents 0 or 1.

Advantageously, the diblock fluorophilic compound has the formula R_{F}LR_{H}, in which R_{F} is a fluorinated alkyl having from 2 to 12 carbon atoms, R_{H} is a saturated or unsaturated, linear, branched or cyclic hydrocarbon-based group having from 2 to 16 carbon atoms, and L is a linker group comprising, for example, a carbon-carbon single bond or an oxygen atom or any other appropriate group, in particular those mentioned above.

Advantageously, the diblock or triblock fluorophilic compound is chosen from the following, where n, m and p are integers:
compounds of formula CₙF₂ₙ₊₁CₘH₂ₘ₊₁ (saturated), or of formula CₙF₂ₙ₊₁CₘH₂ₘ₋₁ (unsaturated), or combinations thereof, n being an integer from 2 to 12 and m being an integer from 2 to 16,
compounds of formula CₚH₂ₚ₊₁—CₙF₂ₙ—CₘH₂ₘ₊₁, with p=1-12, m=1-12 and n=2-12,
compounds of formula CₙF₂ₙ₊₁—CH═CH—CₘH₂ₘ₊₁, with n and m, which may be identical or different, between 2 and 12,
substituted ether or polyether compounds (i.e.: XCₙF₂ₙOCₘH₂ₘX, XCF₂OCₙH₂ₙ OCF₂X, with n and m=1-4, X=Br, Cl or I),
ether diblock or triblock compounds, in particular:
   a) CₙF₂ₙ₊₁—O—CₘH₂ₘ₊₁, with n=2-10; m=2-16,
   b) CₚH₂ₚ₊₁—O—CₙF₂ₙ—O—CₘH₂ₘ₊₁, with p=2-12, m=1-12 and n=2-12.

It is recalled that the nomenclature of the diblock or triblock fluorophilic compounds is known to those skilled in the art. For example, the diblocks FₙHₘ represent the simplified writing of the diblocks CₙF₂ₙ₊₁CₘH₂ₘ₊₁ (with m=8, 12, 14 for 1-octene, 1-dodecene, 1-tetradecene), it being understood that one or more of the hydrogen atoms can be replaced with a halogen, in particular an iodine. For example:
the diblock fluorophilic compound F4H8I has the formula CF₃—(CF₂)₂—CF₂—CH₂—CHI—CH₂—(CH₂)₄—CH₃,
the diblock fluorophilic compound F4H14I has the formula CF₃—(CF₂)₂—CF₂—CH₂—CHI—CH₂—(CH₂)₁₀—CH₃.

The diblock fluorophilic compounds CₙF₂ₙ₊₁CₘH₂ₘ₊₁ are particularly advantageous for the present invention.

The hydrocarbon-based group and/or the fluorinated group of the diblock or triblock fluorophilic compound may also:
comprise phosphorus (for example (perfluoroalkyl)alkylene mono- or dimorpholinophosphate and fluorinated phospholipids),
be substituted with an alcohol, comprise a polyol, or comprise a polyhydroxylated or amino group, be substituted with an amine oxide or amino acid group.

Mention will also be made of the (perfluoroalkyl)alkylene phosphate diblock: R_{F}R₁—OP(O)[N(CH₂CH₂)₂]O₂ or [R_{F}R₁O]₂P(O)[N(CH₂CH₂)₂O], where R_{F} is CF₃(CF₂)ₜ, with t being an integer between 1 and 11, and R1 is a saturated or unsaturated, linear or branched hydrocarbon-based chain, and R_{F} and R₁ can comprise at least one O and/or S atom.

Amphiphilic Lipids

The amphiphilic lipids comprise a hydrophilic part and a lipophilic part. They are generally chosen from compounds in which the lipophilic part comprises a linear or branched, saturated or unsaturated chain having from 8 to 30 carbon atoms. They can be chosen from phospholipids, cholesterols, lysolipids, sphingomyelins, tocopherols, glucolipids, stearylamines, cardiolipins of natural or synthetic origin; molecules composed of a fatty acid coupled to a hydrophilic group via an ether or ester function, such as sorbitan esters, for instance sorbitan monooleate and monolaurate; polymerized lipids; sugar esters, such as sucrose monolaurate and dilaurate, sucrose monopalmitate and dipalmitate or sucrose monostearate and distearate; it being possible for said amphiphilic lipids to be used alone or as a mixture.

Advantageously, the amphiphilic lipid is a phospholipid, preferably chosen from: phosphatidylcholine, dioleoylphosphatidylcholine, dimyristoylphosphatidylcholine, dipalmitoylphosphatidylcholine, distearoylphosphatidylcholine, phosphatidylethanolamine, sphingomyeline, phosphatidylserine and phosphatidylinositol. Egg yolk phosphatidylcholine is a preferred amphiphilic lipid.

According to one particular embodiment, all or part of the amphiphilic lipid may have a reactive function, such as a maleimide, thiol, amine, ester, oxyamine or aldehyde or alkyne or azide group. The presence of reactive functions makes it possible to graft functional compounds at the level of the interface between the aqueous phase and the fluorinated phase.

Pegylated Lipid

In addition to the amphiphilic lipid, to the amphiphilic targeting ligand, to the diblock or triblock fluorophilic compound and to the optional amphiphilic paramagnetic metal chelate, the surfactant may comprise pegylated lipids, i.e. lipids bearing polyethylene oxide (PEG) groups, such as polyethylene glycol/phosphatidylethanolamine (PEG-PE). These pegylated lipids make it possible to act on the stealthy nature of the composition according to the invention in the organism. For the purposes of the present application, the term “polyethylene glycol”, PEG, generally denotes compounds comprising a —CH₂—(CH₂—O—CH₂)ₖ—CH₂OR₃ chain in which k is an integer ranging from 2 to 100 (for example 2, 4, 6, 10, 50) and R₃ is chosen from H, alkyl or —(CO)Alk, the term “alkyl” or “alk” denoting a linear or branched hydrocarbon-based aliphatic group having approximately from 1 to 6 carbon atoms in the chain. The term “polyethylene glycol” as used here encompasses in particular amino polyethylene glycol compounds. Use is in particular made of PEG 350, PEG 750, PEG 2000, PEG 3000 and PEG 5000, modified by adding lipophilic groups in order to insert into the surfactant layer of the nanodroplet, in particular;
1,2-Distearoyl-sn-Glycero-3-Phosphoethanolamine-N-[Methoxy(Polyethylene glycol)-750]
1,2-Distearoyl-sn-Glycero-3-Phosphoethanolamine-N-[Methoxy(Polyethylene glycol)-2000],
1,2-Distearoyl-sn-Glycero-3-Phosphoethanolamine-N-[Methoxy(Polyethylene glycol)-5000].

Use will in particular be made of the pegylated lipid:

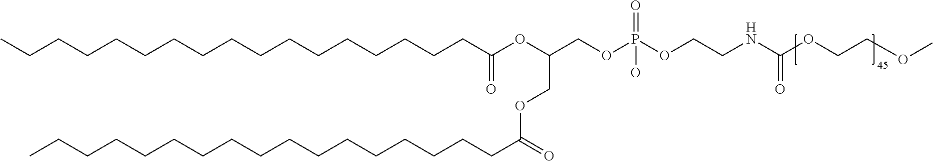

Fluorochromic Amphiphilic Compounds

According to variants, fluorochromic amphiphilic compounds can be integrated in order to combine F19 MRI and fluorescence optical imaging, such as DSPE-rhodamine.

Amphiphilic Targeting Ligand

The nanoemulsions of the applicant are vectorized using at least one amphiphilic targeting ligand, the biological recognition part located at the external surface of the nanodroplets, capable of recognizing the biological target of which the expression is modified in a pathological region (tumor, for example), compared with the healthy region. The nanoemulsion comprises at least one ligand for targeting a pathological region, anchored in the nanodroplet, typically by means of a target-ligand-anchoring group. Advantageously, the number of targeting ligands per nanodroplet is at least 1000 and typically about 1000, 2000, 5000 or 10 000.

As previously indicated, the nanoemulsions obtained have the significant advantage of having increased stability and increased affinity. The applicant explains this advantageous technical effect by the increase in the incorporation of the targeting ligands into the interface formed by the surfactants, between the fluorinated oil and the aqueous phase. The diblock or triblock fluorophilic compounds prove to have the property of a cosurfactant, being located in the fluorinated oil/aqueous phase interface of the droplets. The fluorinated chain of the diblock or triblock fluorophilic compounds is oriented toward the fluorinated core (PFOB oil for example) of the droplet, and the alkylated chain of the diblock or triblock fluorophilic compounds is positioned on the side of the lipid chains of the amphiphilic lipids. According to advantageous embodiments, the alkylated chain of the diblocks or triblocks is chosen so as to have a strong interaction with the lipid chains of the amphiphilic lipids, advantageously by using chains of close or identical length, for example C12, C14 or C16 chains.

The amphiphilic targeting ligands comprise a targeting ligand and a lipophilic group, the whole having an amphiphilic nature. The targeting ligands (targeting ligand part of the amphiphilic targeting ligand) for recognition of the target in a biological medium are grafted, by means of chemical groups, onto a lipophilic part allowing anchoring of the targeting ligand in the layer of surfactants. This enables the targeting ligands to be essentially on the external surface side of the nanodroplets.

Advantageously, the targeting ligand of the amphiphilic targeting ligand (namely the biological recognition part of the amphiphilic targeting ligand, located the external surface of the nanodroplets) is chosen from: pharmacophores, peptides (advantageously of less than 20 amino acids, more advantageously from 5 to 10 amino acids), pseudopeptides, peptidomimetics, amino acids, integrin-targeting agents (peptides and pseudopeptides, peptidomimetic in particular), glycoproteins, lectins, biotin, pteroic or amino pteroic derivatives, folic and antifolic acid derivatives, antibodies or antibody fragments, avidin, steroids, oligonucleotides, ribonucleic acid sequences, deoxyribonucleic acid sequences, hormones, proteins, which are optionally recombinant or mutated, mono- or polysaccharides, compounds with a benzothiazole, benzofuran, styrylbenzoxazole/thiazole/imidazole/quinoline or styrylpiridine backbone and derivative compounds, and mixtures thereof. The peptides, the folic and antifolic acid derivatives, the integrin-targeting agents (peptides and pseudopeptides, peptidomimetics in particular), the cell receptor or enzyme targeting agents (in particular for targeting kinases, in particular tyrosine kinase; metalloproteases; caspases, etc.) are particularly preferred.

The term “pharmacophore” is intended to mean molecules known for their ability to have a pharmacological effect, in particular by virtue of their ability to target at least one biological target (cell receptor, for example).

More globally, according to advantageous embodiments, the targeting ligand of the amphiphilic targeting ligand is chosen from the following list (the documents and references between parentheses are examples and not a limiting list):

1) Targeting ligands targeting VEGF receptors and angiopoietin (described in WO 01/97850), polymers such as polyhistidine (U.S. Pat. No. 6,372,194), fibrin-targeting polypeptides (WO 2001/9188), integrin-targeting peptides (WO 01/77145, WO 02/26776 for alpha v beta3, WO 02/081497, for example RGDWXE), pseudopeptides and peptides for targeting MMP metalloproteases (WO 03/062198, WO 01/60416), peptides targeting, for example, the KDR/Flk-I receptor, including R-X-K-X-H and R-X-K-X-H, or the Tie-1 and 2 receptors (WO 99/40947 for example), Lewis sialyl glycosides (WO 02/062810 and “Müller et al, Eur. J. Org. Chem, 2002, 3966-3973), antioxidants such as ascorbic acid (WO 02/40060), ligands for targeting tuftsin (for example U.S. Pat. No. 6,524,554), for targeting GPCR G-protein receptors, in particular cholecystokinin (WO 02/094873), combinations between integrin antagonist and guanidine mimetic (U.S. Pat. No. 6,489,333), quinolones targeting alpha v beta3 or 5 (U.S. Pat. No. 6,511,648), benzodiazepines and analogs targeting integrins (USA 2002/0106325, WO 01/97861), imidazoles and analogs (WO 01/98294), RGD peptides (WO 01/10450), antibodies or antibody fragments (FGF, TGFb, GV39, GV97, ELAM, VCAM, inducible by TNF or IL (U.S. Pat. No. 6,261,535)), targeting molecules modified by interaction with the target (U.S. Pat. No. 5,707,605), agents for targeting amyloid deposits (WO 02/28441 for example), cathepsin cleaved peptides (WO 02/056670), mitoxantrones or quinones (U.S. Pat. No. 6,410,695), polypeptides targeting epithelial cells (U.S. Pat. No. 6,391,280), cysteine protease inhibitors (WO 99/54317), the targeting ligands described in: U.S. Pat. No. 6,491,893 (GCSF), US 2002/0128553, WO 02/054088, WO 02/32292, WO 02/38546, WO 20036059, U.S. Pat. No. 6,534,038, WO 01/77102, EP 1 121 377, Pharmacological Reviews (52, No. 2, 179; growth factors PDGF, EGF, FGF, etc.), Topics in Current Chemistry (222, W. Krause, Springer), Bioorganic & Medicinal Chemistry (11, 2003, 1319-1341; tetrahydrobenzazepinone derivatives targeting alpha v beta3).

2) Angiogenesis inhibitors, especially those tested in clinical trials or already marketed, especially:
angiogenesis inhibitors involving FGFR or VEGFR receptors such as SU101, SU5416, SU6668, ZD4190, PTK787, ZK225846, azacyclic compounds (WO 02/44156, WO 02/059110);
angiogenesis inhibitors involving MMPs such as BB25-16 (marimastat), AG3340 (prinomastat), solimastat, BAY12-9566, BMS275291, metastat and neovastat;
angiogenesis inhibitors involving integrins such as SM256, SG545, adhesion molecules blocking EC-ECM (such as EMD 121-974 or vitaxin);
medicaments with a more indirect antiangiogenesis mechanism of action such as carboxiamidotriazole, TNP470, squalamine or ZD0101;
the inhibitors described in document WO 99/40947, monoclonal antibodies that are highly selective for binding to the KDR receptor, somatostatin analogs (WO 94/00489), selectin binding peptides (WO 94/05269), growth factors (VEGF, EGF, PDGF, TNF, MCSF, interleukins); VEGF targeting ligands described in Nuclear Medicine Communications, 1999, 20;
the inhibitory peptides of document WO 02/066512.

3) Targeting ligands capable of targeting receptors: CD36, EPAS-1, ARNT, NHE3, Tie-1, 1/KDR, Flt-1, Tek, neuropilin-1, endoglin, pleientropin, endosialin, Axl., alPi, a2ssl, a4P1, a5pl, eph B4 (ephrin), laminin A receptor, neutrophilin 65 receptor, leptin OB-RP receptor, chemokine receptor CXCR-4 (and other receptors cited in document WO 99/40947), LHRH, bombesin/GRP, gastrin, VIP, CCK receptors.

4) Targeting ligands of tyrosine kinase inhibitor type.

5) Known inhibitors of the GPIIb/IIIa receptor chosen from: (1) the fab fragment of a monoclonal antibody of the GPIIb/IIIa, Abciximab receptor, (2) small peptide and peptidomimetic molecules injected intravenously such as eptifibatide and tirofiban.

6) Antagonist peptides of fibrinogen receptors (EP 0 425 212), peptides that are ligands of IIb/IIIa receptors, fibrinogen ligands, thrombin ligands, peptides capable of targeting atheroma plaques, platelets, fibrin, hirudin-based peptides, guanine-based derivatives targeting the IIb/IIIa receptor.

7) Other targeting ligands or biologically active fragments of targeting ligands known to those skilled in the art as medicaments with antithrombotic, anti-platelet aggregation, antiatherosclerotic, antirestenotic or anticoagulant activity.

8) Other targeting ligands or biologically active fragments of targeting ligands targeting alpha v beta3, described in combination with DOTAs in U.S. Pat. No. 6,537,520, chosen from the following: mitomycin, tretinoin, ribomustin, gemcitabin, vincristin, etoposide, cladribin, mitobronitol, methotrexate, doxorubicin, carboquone, pentostatin, nitracrin, zinostatin, cetrorelix, letrozole, raltitrexed, daunorubicin, fadrozole, fotemustin, thymalfasin, sobuzoxane, nedaplatin, cytarabin, bicalutamide, vinorelbin, vesnarinone, aminoglutethimide, amsacrin, proglumide, elliptinium acetate, ketanserin, doxifluridin, etretinate, isotretinoin, streptozocin, nimustin, vindesin, flutamide, drogenil, butocin, carmofur, razoxane, sizofilan, carboplatine, mitolactol, tegafur, ifosfamide, prednimustine, picibanil, levamisole, teniposide, improsulfan, enocitabin, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, leutinizing hormone releasing factor.

9) Certain targeting ligands targeting particular types of cancer, for example peptides targeting the ST receptor associated with colorectal cancer, or the tachykinin receptor.

10) Targeting ligands using phosphine-type compounds.

11) Targeting ligands for targeting P-selectin, E-selectin; for example, the 8-amino-acid peptide described by Morikawa et al., 1996, 951, and also various sugars.

12) Annexin V or targeting ligands targeting apoptotic processes.

13) Any peptide obtained via targeting technologies such as phage display, optionally modified with unnatural amino acids (http//chemlibrary.bri.nrc.ca), for example peptides derived from RGD phage display libraries.

14) Other known peptide targeting ligands for targeting atheroma plaques, cited especially in document WO 2003/014145.

15) Vitamins.

16) Hormone receptor ligands including hormones and steroids.

17) Targeting ligands targeting opioid receptors.

18) Targeting ligands targeting TKI receptors.

19) LB4 and VnR antagonists.

20) Nitriimidazole and benzylguanidine compounds.

21) Targeting ligands recalled in Topics in Current Chemistry, vol. 222, 260-274, Fundamentals of Receptor-based Diagnostic Metallopharmaceuticals, especially:
ligands for targeting peptide receptors overexpressed in tumors (LHRH, bombesin/GRP receptors, VIP receptors, CCK receptors, tachykinin receptors, for example), especially somatostatin or bombesin analogs, optionally glycosylated octreotide-based peptides, VIP peptides, alpha-MSH, CCK-B peptides;
peptides chosen from: RGD cyclic peptides, fibrin-alpha chain, CSVTCR, tuftsin, fMLF, YIGSR (receptor: laminin).

22) Oligosaccharides, polysaccharides and saccharide derivatives, derivatives targeting the Glut receptors (saccharide receptors).

23) Targeting ligands used for smart-type products.

24) Myocardial viability markers (tetrofosmine and hexakis(2-methoxy-2-methylpropylisonitrile)).

25) Sugar and fat metabolism tracers.

26) Neurotransmitter receptor ligands (receptors D, 5HT, Ach, GABA, NA).

27) Oligonucleotides.

28) Tissue factor.

29) Targeting ligands described in WO 03/20701, in particular the PK11195 ligand of the peripheral benzodiazepine receptor.

30) Fibrin-binding peptides, especially the peptide sequences described in WO 03/11115.

31) Amyloid plaque aggregation inhibitors (described, for example, in WO 02/085903).

32) Pharmacophore compounds for targeting Alzheimer's disease, in particular compounds comprising backbones of benzothiazole, benzofuran, styrylbenzoxazole/thiazole/imidazole/quinoline, styrylpyridine type.

33) Integrin-targeting compounds in particular having an affinity greater than 10 000, 100 000 or more, in particular non-peptide compounds which are mimetics of RGD peptides, and in particular compounds comprising a tetrahydronaphthyridine group, described for example in: J Med. Chem., 2003, 46, 4790-4798, Bioorg. Med. Chem. Letters, 2004, 14, 4515-4518, Bioorg. Med. Chem. Letters, 2005, 15, 1647-1650.

In particular, for these naphthyridine compounds, the applicant uses any naphthyridine compound known in the prior art (in particular those of WO 2009/114776), the use of naphthyridine compounds as a targeting ligand for medical imaging being described in WO 2007/042506 on page 13, lines 30-34.

Alternatively or cumulatively, the amphiphilic targeting ligands are advantageously written in the form:

Bio-L₁-L₂-Lipo

in which:
Bio is a targeting ligand, in particular chosen from those mentioned above (i.e. the biological recognition part, which is located at the external surface of the nanodroplets);
Lipo is a lipophilic group for inserting the targeting ligand into the surfactant layer;
L₂ is a linker group, advantageously chosen from:
   C₁₋₆ alkylene, PEG, for example CH₂—(CH₂—O—CH₂)ₖ—CH₂ with k=2 to 10, (CH₂)₃—NH, NH—(CH₂)₂—NH, NH—(CH₂)₃—NH, nothing or a single bond, (CH₂)ₙ, (CH₂)ₙ—CO—, —(CH₂)ₙNH—CO— with n being an integer from 2 to 10, (CH₂CH₂O)_{q}(CH₂)ᵣ—CO—, (CH₂CH₂O)_{q}(CH₂)ᵣ—NH—CO— with q being an integer from 1 to 10 and r an integer from 2 to 10, (CH₂)ₙ—CONH—, (CH₂)ₙ—CONH-PEG, (CH₂)ₙ—NH—HOOC—CH₂—O—(CH₂)₂—O—(CH₂)₂—O—CH₂—COOH; HOOC—(CH₂)₂—CO₂—(CH₂)₂—OCO—(CH₂)₂—COOH; HOOC—CH(OH)—CH(OH)—COOH; HOOC—(CH₂)ₙ—COOH; NH₂—(CH₂)ₙ—NH₂, with n being an integer from 0 to 20; NH₂—(CH₂)ₙ—CO₂H; NH₂—CH₂—(CH₂—O—CH₂)ₙ—CO₂H with n being an integer from 1 to 10, squarate
P1-I-P2, P1 and P2, which may be identical or different, being chosen from O, S, NH, nothing (single bond), CO₂, NHCO, CONH, NHCONH, NHCSNH, SO₂NH—, NHSO₂—, squarate
with I=alkylene, alkoxyalkylene, polyalkoxyalkylene (in particular PEG), alkylene interrupted with one or more squarates or with one or more aryls, advantageously phenyl, alkenylene, alkynylene, alkylene interrupted with one or more groups chosen from —NH—, —O—, —CO—, —NH(CO)—, —(CO)NH—, —O(CO)—, or —(OC)O—,
L₁ is chosen from a single bond, —CONH—, —COO—, —NHCO—, —OCO—, —NH—CS—NH—, —C—S—, —N—NH—CO—, —CO—NH—N—, —CH₂—NH—, —N—CH₂—, —N—CS—N—, —CO—CH₂—S—, —N—CO—CH₂—S—, —N—CO—CH₂—CH₂—S—, —CH═NH—NH—, —NH—NH═CH—, —CH═N—O—, —O—N═CH—, or corresponds to the following formulae:

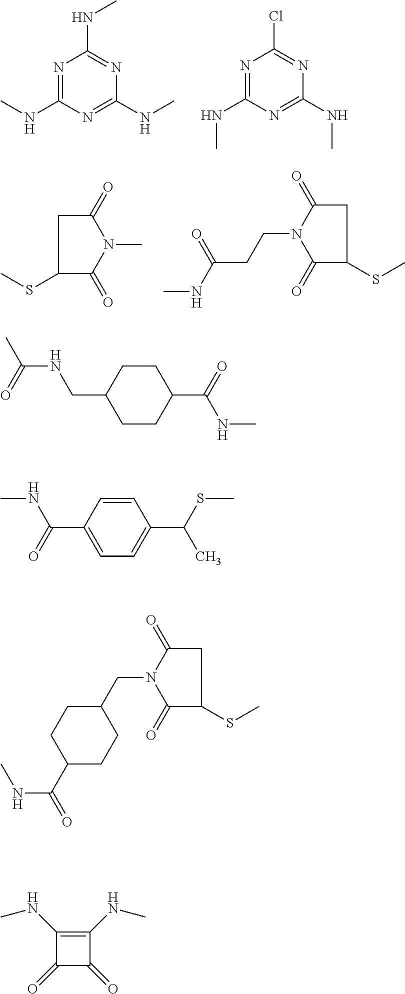

A number of examples of peptide or pharmacophore targeting ligands made amphiphilic for anchoring at the external surface of the nanodroplet (hereinafter, peptide targeting ligands, folic acid derivatives, naphthyridine derivatives) are presented.

The application presents illustrative and nonlimiting examples of their synthesis.

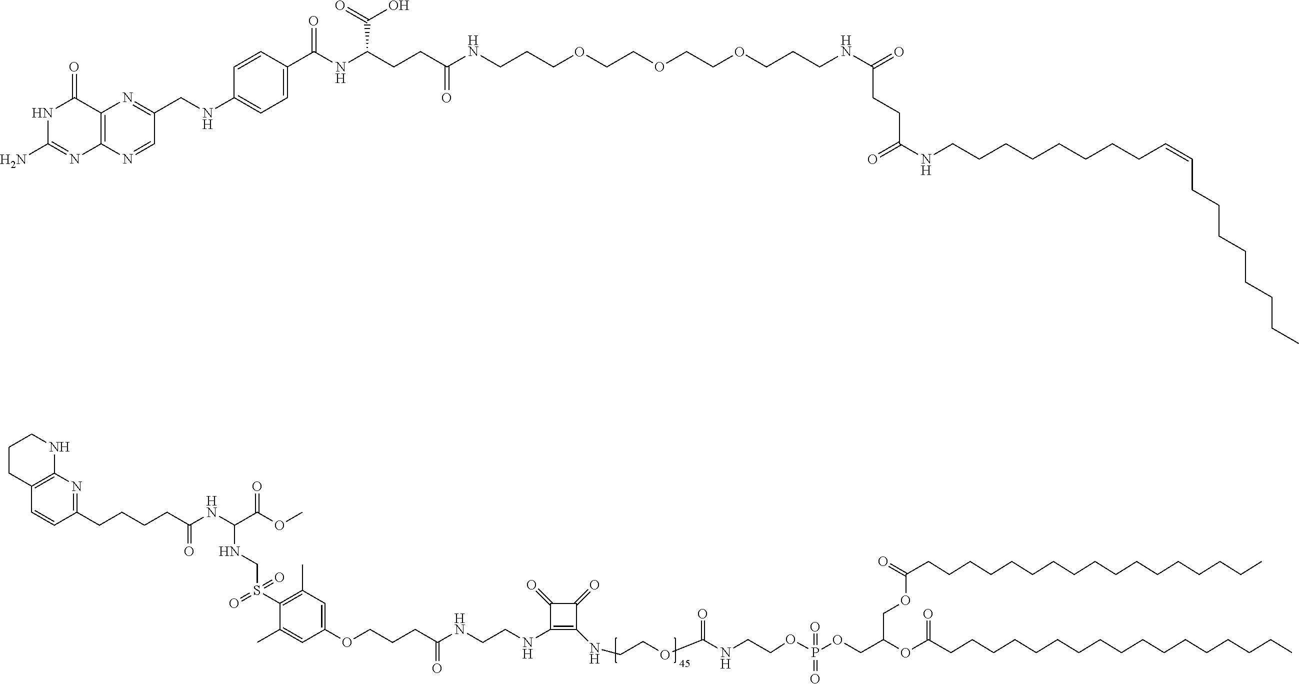

Amphiphilic Paramagnetic Metal Chelate

According to embodiments, the nanoemulsion comprises an amphiphilic paramagnetic metal chelate, which is preferably macrocyclic, comprising a hydrophilic core chosen from: DOTA, DO3A, HPDO3, BTDO3A, PCTA, DOTAM, DOTMA, DOTA-GA and derivatives thereof. Advantageously, the hydrophilic part of the amphiphilic chelate is a macrocyclic chelate chosen from: DOTA, DO3A, HPDO3, BTDO3A, PCTA and any known derivative of these chelates, in particular described, for example, in Mini Reviews in Medicinal Chemistry, 2003, vol. 3, No. 8. The chelate is made amphiphilic by chemically modifying it, typically by coupling to one or more fatty chains, so as to exhibit lipophilicity (sufficiently high lipophilicity or, conversely, sufficiently low hydrophilicity), such that it can be anchored in the surfactant layer of the nanodroplet and so as to form a lipid composition which is sufficiently stable for satisfactory diagnostic use. There will for example be, in a nonlimiting manner, a choice of the amphiphilic groups grafted to the chelate such that the HLB (hydrophilicity/lipophilicity balance) value of the chelate is about 13 to 20 for the chelates anchored to the nanoemulsions. Advantageously, the number of lanthanide chelates per nanodroplet is at least 1000 and typically at least 5000, 10 000, 20 000, 50 000 to 100 000.

Fluorinated Oil

Any appropriate fluorinated oil (and/or mixture of fluorinated oils) can be used, including perfluorocarbons which are linear or branched, or cyclic or polycyclic, and saturated or unsaturated, perfluorinated cyclic tertiary amines, perfluoro esters or thioesters, haloperfluorocarbons and known analog or derivative compounds. Advantageously, at least 60% of the hydrogen atoms of the corresponding hydrocarbon-based oil are replaced with a fluorine atom. Typically, these fluorinated oils are chains of 2 to 16 atoms, perfluoroalkanes, bis(perfluoroalkyl)alkenes, perfluoroethers, perfluoroamines, perfluoroalkyl bromides, or perfluoroalkyl chlorides. According to advantageous embodiments, the lipid nanodroplet includes perfluorocarbons as described in U.S. Pat. No. 5,958,371, the liquid emulsion containing nanodroplets comprising a perfluorocarbon with quite a high boiling point (for example between 30 and 90° C., preferably between 50 and 150° C., for example 142° C. for PFOB), surrounded by a coating composed of a lipid and/or of a surfactant.

The perfluorinated oils for perfluorocarbon emulsions for MRI imaging are recalled in particular in documents U.S. Pat. No. 6,676,963, U.S. Pat. No. 4,927,623, U.S. Pat. No. 5,077,036, U.S. Pat. No. 5,114,703, U.S. Pat. No. 5,171,755, U.S. Pat. No. 5,304,325, U.S. Pat. No. 5,350,571, U.S. Pat. No. 5,393,524, and U.S. Pat. No. 5,403,575; in particular the oils: perfluorooctylbromide PFOB, C₈F₁₇Br (PFOB or perfluorobron), perfluorooctylethane (C₈F₁₇C₂H₅ PFOE), perfluorodecalin FDC, perfluorooctane C₈F₁₈, perfluorodichlorooctane, perfluoro-n-octyl bromide, perfluoroheptane, perfluorodecane C₁₀F₂₂, perfluorododecyl bromide C₁₀F₂₂Br PFDB, perfluorocyclohexane, perfluoromorpholine, perfluorotripropylamine, perfluorotributylamine, perfluorodimethylcyclohexane, perfluorotrimethylcyclohexane, perfluorodicyclohexyl ester, perfluoro-n-butyltetrahydrofuran.

Included in the definition of the fluorinated oils are the oils of formula CₙF₂ₙ₊₁X, XCₙF₂ₙX, where n is an integer ranging from 2 to 10, X=Br, Cl or I,

in particular: 1-bromo-F-butane (n-C₄F₉Br), 1-bromo-F-hexane (n-C₆F₁₃Br), 1-bromo-F-heptane (n-C₇F₁₅Br), 1,4-dibromo-F-butane and 1,6-dibromo-F-hexane.

Also included are fluorinated compounds with chlorinated substituents, for example: perfluorooctyl chloride (n-C₈F₁₇Cl), 1,8-dichloro-F-octane (n-ClC₈F₁₆Cl), 1,6-dichloro-F-hexane (n-ClC₆F₁₂Cl) and 1,4-dichloro-F-butane (n-ClC₄F₈Cl).

Also included are the fluorinated oils of formula CₙF₂ₙ₊₁OCₘF₂ₘ₊₁, CₙF₂ₙ₊₁CH═CHCₘF₂ₘ₊₁, for example: C₄F₉CH═CHC₄F₉ (F-44E), i-C₃F₉CH═CHC₆F₁₃ (F-i36E), and C₆F₁₃CH═CHC₆F₁₃ (F-66E), where n and m are identical or different, and are integers between 2 and 12.

Also included are polycyclic or cyclic compounds such as: C₁₀F₁₈ (F-decalin or perfluorodecalin), and mixtures of perfluoroperhydrophenanthrene and perfluoro-n-butyldecalin.

Also included are perfluorinated amines, such as: F-tripropylamine (“FTPA”), F-tributylamine (“FTBA”), F-4-methyloctahydroquinolizine (“FMOQ”), F—N-methyl-decahydroisoquinoline (“FMIQ”), F—N-methyldecahydroquinoline (“FHQ”), F—N-cyclohexylpyrrolidine (“FCHP”) or F-2-butyltetrahydrofuran (“FC-75” or “FC-77”).

Among the mixtures of fluorinated oils, mention will be made of the mixtures of from 10% to 70% of a first oil, of PFOB type for example, and of 30% to 70% of a second fluorinated oil.

Aqueous Phase

The aqueous phase is advantageously water or a pharmaceutically acceptable aqueous solution such as a saline solution or a buffer solution.

Proportions of the Constituents of the Composition

More specifically, the oil-in-water nanoemulsion composition comprises, according to advantageous embodiments:
an aqueous phase, preferably representing 29.4% to 80% by weight of the composition, advantageously 55% to 65%, more advantageously from 58% to 62%,
a fluorinated phase comprising at least one fluorinated oil, representing 19.4% to 70% by weight of the composition, advantageously 35% to 45%, more advantageously 37% to 42%,
a surfactant (forming the surfactant layer) at the interface between the aqueous and fluorinated phases, the surfactant comprising at least one diblock or triblock fluorophilic compound, at least one amphiphilic lipid and at least one amphiphilic targeting ligand,
the total surfactant content by weight relative to the fluorinated oil being between 3% and 15%, in particular between 3% and 12%, advantageously between 4% and 8%,
the total surfactant content by weight relative to the composition being between 0.6% and 7%, advantageously between 1% and 3%.

Advantageously, the amphiphilic targeting ligand represents 0.1 mol % to 10 mol % of the total surfactants, advantageously 0.5% to 5%, in particular 1% to 2%.

Advantageously, the surfactant comprises:
50 mol % of diblock or triblock fluorophilic compounds,
50 mol % of other surfactants (i.e. non-fluorinated amphiphilic compounds).

Advantageously, the 50% of other surfactants (non-fluorinated amphiphilic compounds) comprise:
50 mol % to 95 mol % of amphiphilic lipid,
0 mol % to 25 mol % of amphiphilic paramagnetic metal chelate,
0.1 mol % to 10 mol % of amphiphilic targeting ligand,
0 mol % to 10 mol % of pegylated lipids,
0.1 mol % to 0.5 mol % of amphiphilic compounds comprising a fluorophore (for example, rhodamine).

Advantageously, for a fluorinated phase representing 19.4% to 70%, and in particular 30% to 50%, of the composition, the weight content of total surfactant relative to the fluorinated phase is greater than 3%, preferably from 3% to 8%, more preferably from 4% to 6%.

Preferably, the surfactant of the composition according to the invention comprises:
non-fluorinated amphiphilic compounds, of which 80 mol % to 95 mol % of amphiphilic lipid, 0 mol % to 5 mol % of pegylated lipids and 0.1 mol % to 10 mol % of amphiphilic targeting ligand,
diblock or triblock fluorophilic compounds;
   the non-fluorinated amphiphilic compounds representing 30 mol % to 60 mol % of the total surfactants, and the diblock or triblock fluorophilic compounds representing 30 mol % to 60 mol % of the total surfactants;
   advantageously, the non-fluorinated amphiliphic compounds representing 50 mol % of the total surfactants, and the diblock or triblock fluorophilic compounds representing 50 mol % of the total surfactants.

According to preferred embodiments, the nanoemulsion according to the invention has the following composition by weight:
1) 29.4% to 80% by weight of aqueous phase, advantageously 55% to 65%, more advantageously from 58% to 62%,
2) 19.4% to 70% by weight of fluorinated phase comprising a fluorinated oil, advantageously 35% to 45%, more advantageously 37% to 42%,
3) 0.6% to 7% of total surfactant or else 3% to 10% relative to the fluorinated phase, the surfactant comprising:
   non-fluorinated amphiphilic compounds, of which 80 mol % to 95 mol % of amphiphilic lipid, 0 mol % to 5 mol % of pegylated lipids and 0.1 mol % to 10 mol % of amphiphilic targeting ligand, and
   diblock or triblock fluorophilic compounds.

Preferably, the non-fluorinated amphiphilic compounds represent 30 mol % to 60 mol % of the total surfactants, and the diblock or triblock fluorophilic compounds represent 30 mol % to 60 mol % of the total surfactants (the total of the non-fluorinated amphiphilic compounds and of the diblock or triblock fluorophilic compounds being 100%).

Preferably, the non-fluorinated amphiphilic compounds represent 50 mol % of the total surfactants, and the diblock or triblock fluorophilic compounds represent 50 mol % of the total surfactants.

In particular, among the embodiments below, the following embodiments are advantageous:

| | | | % by weight |
|---|---|---|---|
| | | % by weight of | of surfactant |
| % by weight of | % by weight of | surfactant | relative to the total |
| aqueous phase | oil | relative to the oil | composition |
| (1) | (2) | (3) | (4) |
| 50-70 | 29.1-50 | 3 to 10% of (2) | 0.9-5% (*) |
| 55-65 | 33.95-45 | 3 to 10% of (2) | 1.05-4.5% |
| 57-61 | 37-41 | 3 to 10% of (2) | 1.11-4.1% |
| 55-65 | 33.6-45 | 4 to 8% of (2) | 1.4-3.6% |
| 57-61 | 37-41 | 4 to 8% of (2) | 1.48-3.28% |

It being specified that the total (1) + (2) + (4) = 100%
(*) the range [0.9-5] corresponds to 0.03 × 30 = 0.9% and 0.1 × 50 = 5

These range are preferred in particular since they make it possible to obtain a nanodroplet size of between 150 and 350 nm, and in particular around 200 to 250 nm.

The size and the stability of the nanodroplets are very satisfactory, as is the viscosity (about 2 to 3 mPa·s). They have a Newtonian behavior, which constitutes a considerable advantage for injectable pharmaceutical solutions.

The constituent proportion ranges are, for example, as follows.

| | Embodiment A | Embodiment B | Embodiment C |
|---|---|---|---|
| Aqueous phase of the composition | 29.4 to 80, | 29.4 to 80, | 29.4 to 80, |
| | preferably | preferably | preferably |
| | 55 to 65, | 55 to 65, | 55 to 65, |
| | preferably | preferably | preferably |
| | 59 | 59 | 59 |
| Fluorinated phase as | 19.4 to 70, | 19.4 to 70, | 19.4 to 70, |
| % of the composition | preferably | preferably | preferably |
| | 35 to 45, | 35 to 45, | 35 to 45, |
| | preferably | preferably | preferably |
| | 39 | 39 | 39 |
| Content (mol %) | 3 to 10, | 3 to 10, | 3 to 10, |
| of surfactants relative to the fluorinated | preferably | preferably | preferably |
| phase | 4 to 8 | 4 to 8 | 4 to 8 |
| Diblock or triblock fluorophilic compound | 40 to 60%, | 40 to 60%, | 40 to 60%, |
| content (mol %) | preferably | preferably | preferably |
| of the surfactants | 50% | 50% | 50% |
| Non-fluorinated amphiphilic compound | 40 to 60%, | 40 to 60%, | 40 to 60%, |
| content (mol %) of the surfactants | preferably | preferably | preferably |
| | 50% | 50% | 50% |
| Amphiphilic lipid content (mol %) of the | 60 to 95 | 50 to 95 | 0 |
| non-fluorinated amphiphilic compounds | | | |
| (a) | | | |
| Amphiphilic paramagnetic metal chelate | 0 to 25 | 0 to 25 | 0 to 99.95 |
| content (mol %) of the non-fluorinated | | | |
| amphiphilic compounds (b) | | | |
| Pegylated lipid content (mol %) of the | 0 | 5 to 15 | 0 to 5 |
| non-fluorinated amphiphilic compounds | | | |
| (c) | | | |
| Content (mol %) of amphiphilic | 0.1 to 0.5% | 0.1 to 0.5% | 0.1 to 0.5% |
| compound comprising a fluorophore, of | | | |
| the non-fluorinated amphiphilic | | | |
| compounds (d) | | | |
| Amphiphilic targeting ligand content | 0.1 to 10, | 0.1 to 10, | 0.1 to 10, |
| (mol %) of the non-fluorinated | preferably from | preferably | preferably |
| amphiphilic compounds (e) | 0.5 to 3% | from | from |
| | | 0.5 to 3% | 0.5 to 3% |

In the embodiments of the table above, in the [amphiphilic lipids, amphiphilic paramagnetic metal chelates, pegylated lipids, amphiphilic targeting ligands] collection corresponding to the non-fluorinated amphiphilic compounds, the total (a)+(b)+(c)+(d)+(e) is 100%.

Preparation Process

It is recalled that emulsions are heterogeneous lipid mixtures appropriately obtained in particular by mechanical stirring and/or addition of emulsifiers.

For example, the [amphiphilic targeting ligand/amphiphilic lipid/optional amphiphilic paramagnetic metal chelates/optional pegylated lipids/optional amphiphilic compounds comprising a fluorophore] are dispersed in the aqueous phase with magnetic stirring and using ultrasound. The diblock or triblock fluorophilic compound is then added to this aqueous phase and then the fluorinated oil is introduced dropwise as a preemulsifier using an Ultraturrax for example. The preemulsion is then finalized using a microfluidizer and filtered through 0.45 μm.

Use of the Composition According to the Invention

According to a second aspect, the invention also relates to a contrast agent comprising the composition as described above.

The amphiphilic targeting ligands cited are essentially for diagnostic purposes. The composition according to the invention is therefore of use for diagnosis, in particular by magnetic resonance imaging (MRI). However, nanoemulsions for therapeutic treatment can be prepared. The nanodroplets will then comprise, on the one hand, an amphiphilic targeting ligand anchored in the surfactant layer making it possible to reach the biological target (the pathological region), in particular as defined above, and, on the other hand, an active ingredient used as a medicament for the therapeutic treatment and encapsulated in the droplets of fluorinated oil in the case of active ingredients that are soluble in the fluorinated oil.

It is specified that, given in particular the volume that can be injected to patients, of about 10 to 50 ml, the fluorinated oil is generally used at a sufficiently high content, of at least 20% relative to the total weight of the composition, to have a sufficiently concentrated solution and a sufficient MRI signal. It is important to have a concentration that is suitable for the duration of injection, the moment at which the signal is acquired and the associated processing of the data by the practitioner. If a solution is too dilute, this would generally make it unusable for medical imaging examinations.

The compositions (nanoemulsions) of the applicant have a droplet size which is sufficiently small to allow the latter to circulate in biological media without product degradation, as far as the target of the targeting ligand attached to the droplets. The size is typically from 50 to 400 nm, advantageously 100 to 350 nm, especially 150 to 300 nm, and in particular 200 to 250 nm.

The nanodroplets each comprise a number of targeting ligands of about 100 to 5000, in particular 500 to 2000, which allows effective targeting according to the affinity and the multivalence of the targeting ligand. The biological results obtained by virtue of the novel nanoemulsions of the applicant show, in addition, that the targeting ligands are advantageously distributed over the whole of the external surface of the nanodroplets, which is reflected by optimized multivalence of the targeting ligands.

The amphiphilic targeting ligands advantageously represent 0.1 mol % to 10 mol % of the total surfactants, advantageously 0.5% to 5%, in particular 0.5% to 3%. The injected contrast product having the described nanoemulsion compositions has an affinity advantageously of about 1 pM to 100 nM, in particular 1 pM to 10 nM, advantageously 1 pM to 1 nM (the affinity per amphiphilic targeting ligand is multiplied by the number of targeting ligands per nanodroplet).

Advantageously, the composition comprises 0.001% to 0.1% by weight of amphiphilic targeting ligand, in particular 0.01% to 0.1%. The nanoemulsions of the applicant also have the advantage of being able to control the type and the amount of targeting ligands, and in particular of being able to incorporate different targeting ligands. For example, a nanodroplet will comprise:
an amphiphilic targeting ligand which allows access to a pathological physiological region, for example a targeting ligand for crossing the BBB (blood-brain barrier),
another amphiphilic targeting ligand for targeting which subsequently enables the targeting of a target biological marker overexpressed by certain cells of this pathological region.

The molecular interactions between the targeting ligand and the targeting biological marker enable the nanodroplets to be taken up in the pathological region, and the MRI imaging which results therefrom makes it possible to precisely locate the pathological region.

The composition forming the contrast agent is preferably administered intravascularly, depending on the patient examined.

The lipid compositions obtained are, as appropriate, formulated using known additives summarized, for example, in U.S. Pat. No. 6,010,682, in particular for administration by intravenous injection. Mention will in particular be made of thickeners, saccharides or polysaccharides, glycerol, dextrose, sodium chloride and antimicrobial agents.

Advantageously, by virtue of the compositions according to the invention, it is possible to typically obtain the following characteristics, which can vary depending on the precise compositions of the emulsions and the process for the preparation thereof:
kinematic viscosity (cSt): 1 to 4
osmomality (miliosmol): 250 to 350
number of targeting ligands: 50 to 1000, in particular 100 to 3000
diameter: 10 to 300 nm.

The invention also relates to:
a contrast product, preferably for MRI, comprising the compositions of nanoemulsions of the application,
the nanoemulsions of the applicant for use thereof in diagnosis, in particular in the diagnosis of diseases, in particular cancer diseases, inflammatory diseases, neurodegenerative diseases and cardiovascular diseases.

The invention is illustrated by means of the following examples.

### EXAMPLE 1

### Synthesis of a Linear RGD Lipophile (Amphiphilic Targeting Ligand)

### Step 1

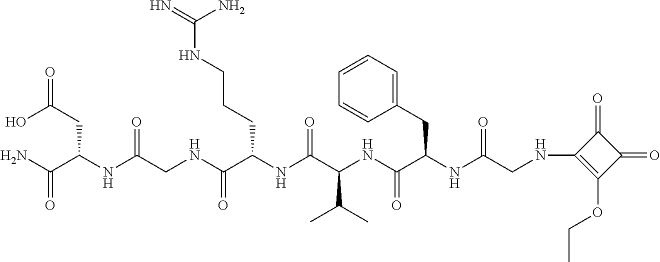 [SMILES: CCOc1c(NCC(=O)N[C@H](Cc2ccccc2)C(=O)N[C@H](C(=O)N[C@@H](CCCNC(=N)N)C(=O)NCC(=O)N[C@@H](CC(=O)O)C(N)=O)C(C)C)c(=O)c1=O]

100 mg (0.15 mmol) of H-Gly-(D)-Phe-(L)-Val-(L)-Arg-Gly-(L)-Asp-NH₂ (H-GfVRGD-NH₂) peptide purchased from Bachem are dissolved under argon in 3 ml of DMSO dried over sieves. 23 μl of 3,4-diethoxy-3-cyclobutene-1,2-dione (0.15 mmol; 1 eq.) and 25 μl of triethylamine are added. The reaction medium is left overnight at 40° C. before being precipitated from 40 ml of diethyl ether. After filtration, 98 mg of a white powder are obtained (yield: 84%).

C₃₄H₄₈N₁₀O₁₁; m/z=773 (ES+)

### Step 2

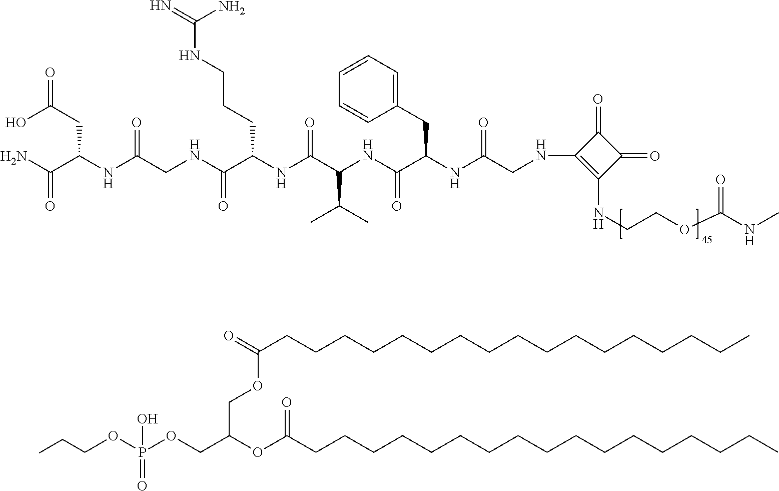

95 mg of the intermediate obtained in a) (0.12 mmol; 1 eq.) and 430 mg (0.15 mmol, 1.25 eq.) of 1,2-distearoyl-sn-glycero-3-phosphoethanolamine-N-[amino(polyethylene glycol)-2000] (ammonium salt) are dissolved in 3 ml of DMSO dried over molecular sieves in the presence of 25 μl of triethylamine. The reaction medium is stirred for 48 h at ambient temperature before being precipitated from 40 ml of diethyl ether. After filtration, 400 mg of a white powder are obtained. The product thus obtained is then purified by flash chromatography on a C4 cartridge with a gradient of 10 mM pH6 ammonium formate/methanol. 260 mg of a white powder are obtained (yield: 62%).

C₁₆₄H₃₀₅N₁₂O₆₄P; MALDI-TOF: Positive mode m/z=3501

### EXAMPLE 2

### Synthesis of a Cyclic RGD Lipophile (Amphiphilic Targeting Ligand)

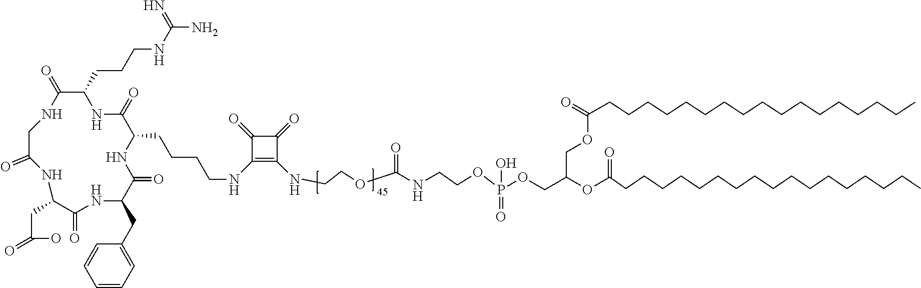

Same procedure as for example 4, starting from 90 mg of cyclic RGDfK peptide purchased from Bachem.

C₁₆₃H₃₀₂N₁₁O₆₃P; MALDI-TOF: Positive mode m/z=3456

### EXAMPLE 3

### Synthesis of a Lipophile with an RGD Peptidomimetic Comprising a Naphthyridine Group (Amphiphilic Targeting Ligand)

### Synthesis Scheme:

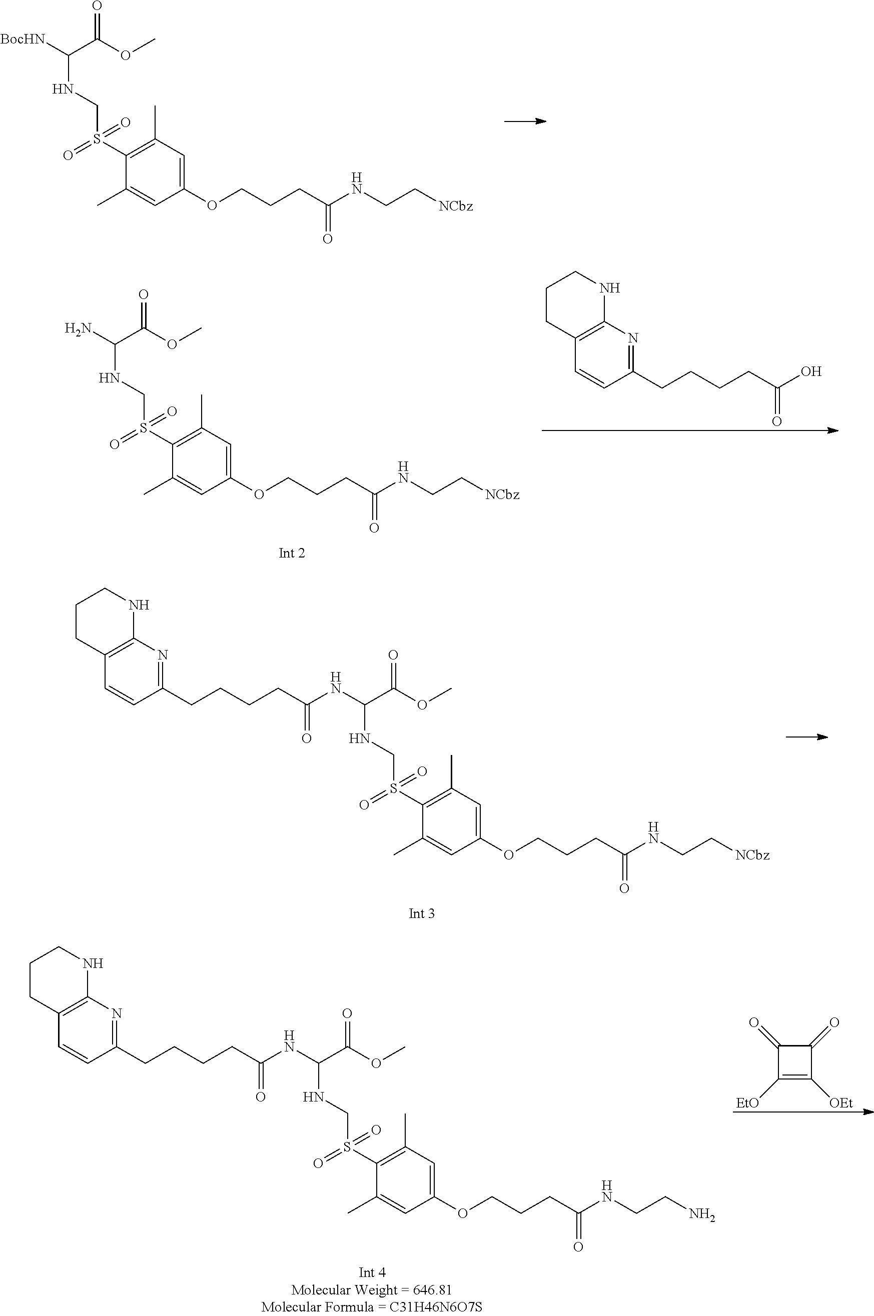

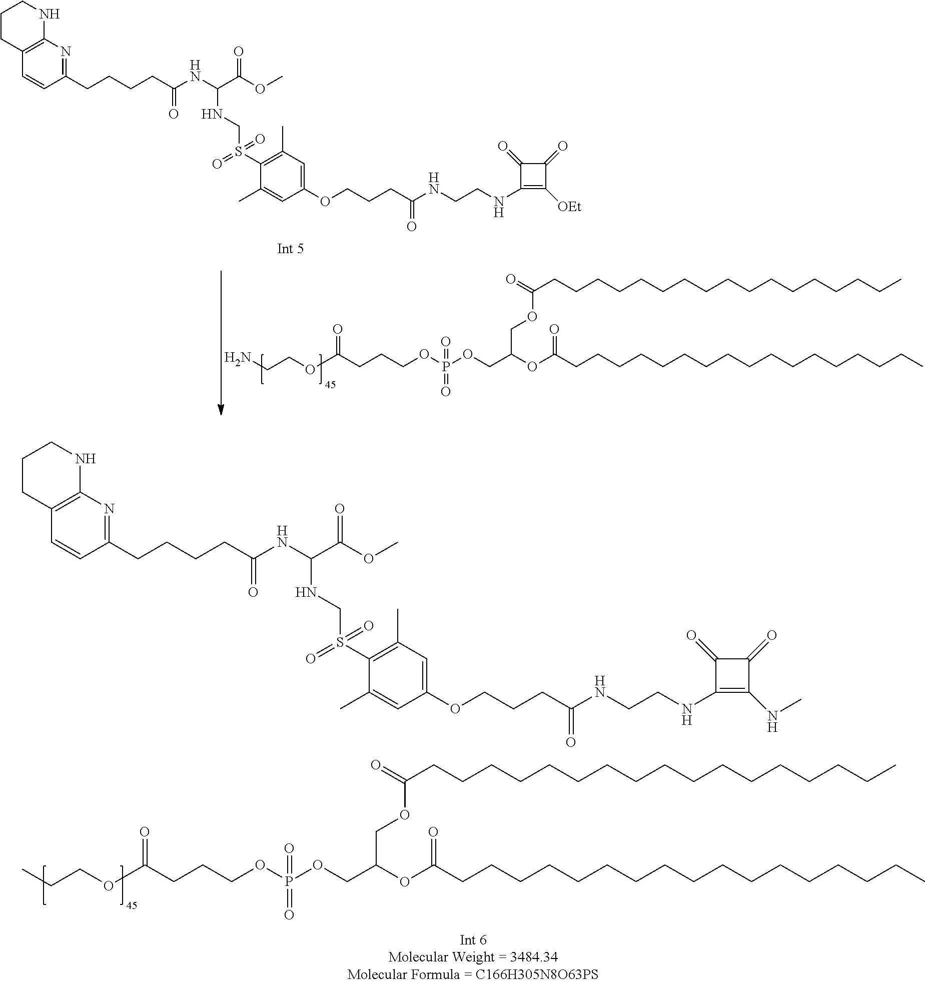

### First Step:

1 g of Int 1 is dissolved in 5 ml of CH₂Cl₂. 5 ml of TFA are added to the medium. The mixture is left for 3 h at ambient temperature and then evaporated to dryness. The residue is taken up in 2×40 ml of iso-ether, and an oil is recovered, which is dried by evaporation.

M_{obt}=0.8 g; Yld=90%; MALDI-TO: Positive mode m/z=564

### Second Step:

| Reagents | Amounts | Solvents |
|---|---|---|
| Int 2 641.031 | M = 0.564 g (0.001 m) | DMF V = 10 ml |
| Sub-contracted | M = 0.235 g (0.00023 m) | |
| naphthyridine | | |
| HOBT | M = 0.131 g | |
| DIPEA | M = 0.286 g | |
| EDCI | V = 0.2 ml | |

Int3 641.032

The acid is dissolved in DMF, HOBT and EDCI are then added and the mixture is left for 1 hour under argon.

Int 2 and DIPEA are added; the mixture is left for 18 h at ambient temperature under argon. After evaporation, the oil is taken up in CH₂Cl₂ and washed with a dilute Na₂CO₃ solution; after evaporation, an oil is obtained.

M_{obt}=0.600 g; Yld=77%; M/Z=780

### Third Step:

| Reagents | Amounts | Solvents |
|---|---|---|
| Int 3 641.032 | M = 0.6 g (0.0077m) | MeOH V = 30 ml |
| Pd/C 10% | 1 spatula-full | |

Int4: 641.034

Int 3 is dissolved in methanol and the solution is placed in a 125 ml autoclave; the catalyst is added and the mixture is left for 3 h under hydrogen pressure (P=5 bar).

After filtering off the catalyst and evaporating, an oil is obtained, which is washed with 50 ml of iso-ether.

Attention should be paid to the possible absorption of the amine onto the catalyst (work with the minimum amount of catalyst).

M_{obt}=0.300 g; Yld=60%; HPLC=90%; M/Z=646

### Fourth Step:

| Reagents | Amounts | Solvents |
|---|---|---|
| Int 4 641.034 | M = 0.300 g | DMSO V = 10 ml |
| | (0.000442 m) | TEA V = 0.25 ml |
| Diethyl squarate | M = 0.286 g | |

Int5: 641.037

Int 4 is dissolved in DMSO and then the diethyl squarate and the TEA are added; the mixture is left overnight at ambient temperature under argon. It is poured into ether: a white paste is obtained.

M_{obt}=0.330 g; Yld=97%; M/Z=770

### Fifth Step:

| Reagents | Amounts | Solvents |
|---|---|---|
| Int 5 641.037 | M = 0.330 g (0.00043 m) | DMSO V = 10 ml |
| DSPE-PEG₂₀₀₀ | M = 1.07 g (0.000385 m) | |
| Saturated Na₂CO₃ solution | M = 0.131 g | |

Int6: 641.040

Int 5 is dissolved in DMSO and 3 drops of saturated Na₂CO₃ solution are added=>solution A.

The DSPE is introduced into 5 ml of DMSO (partial solution): 1 ml of water is added=>always a white cloudiness.

The DSPE solution is introduced into the solution A: a cloudy medium is obtained which is stirred for 24 h. 1.5 ml of water are added and the mixture is left for a further 24 h at ambient temperature: the medium becomes homogeneous.

After evaporating off the water, the residue is washed with 3×50 ml of ether (which makes it possible to remove the DMSO). The paste obtained is dissolved in 50 ml of CH₂Cl₂ and the cloudiness is eliminated by filtration.

The product is purified using a silica, eluting with CH₂Cl₂ (a fine fractionation is carried out). After combining and evaporating the correct fractions, crystals are obtained.

Comment:

The product obtained is in the acid form by cleavage of the methyl ester due to the presence of Na₂CO₃=>Confirmation by MALDI.

M_{obt}=0.170 g; Yld=17%; M/Z=3484 (METHYL ester MW=3498)

### EXAMPLE 4

### Synthesis of an Amphiphilic Gd Complex (Amphiphilic Paramagnetic Metal Chelate)

### Synthesis Scheme:

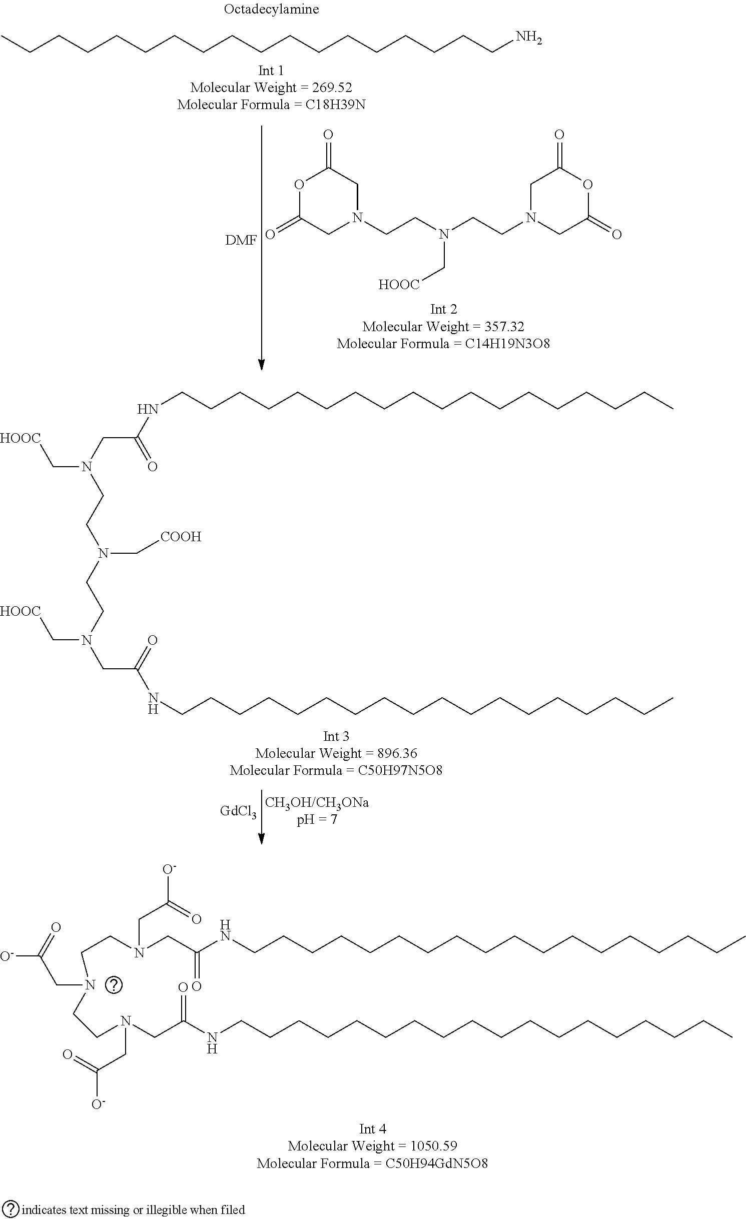

### Opening of DTPA Bisanhydride by Octadecylamine:

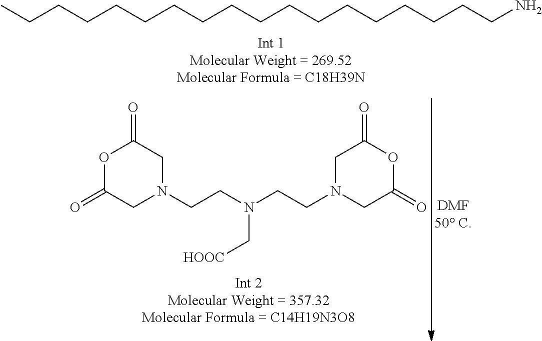 [SMILES: CCCCCCCCCCCCCCCCCCN ; O=C(O)CN(CCN1CC(=O)OC(=O)C1)CCN1CC(=O)OC(=O)C1]

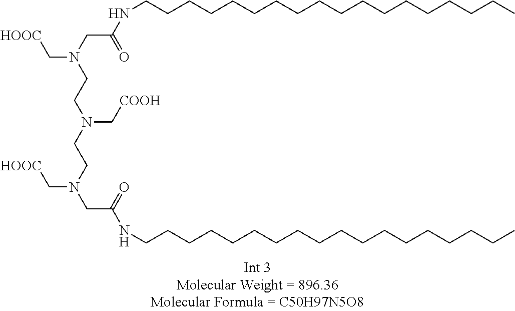 [SMILES: CCCCCCCCCCCCCCCCCCNC(=O)CN(CCN(CCN(CC(=O)O)CC(=O)NCCCCCCCCCCCCCCCCCC)CC(=O)O)CC(=O)O]

| | M (g/mol) | m (g) | n (mmol) | eq | V (mL) |
|---|---|---|---|---|---|
| octadecylamine | 269.52 | 10 | 37.2 | 2.2 | |
| DTPA | 357.32 | 6 | 16.8 | 1 | |
| bisanhydride | | | | | |
| DMF | | | | | 240 |

The DTPA bisanhydride is suspended in the DMF. The suspension is heated to 50° C. and dissolution takes place. The octadecylamine is added in a single portion. The reaction is maintained at 50° C. overnight. During the reaction, the amine is seen to slowly dissolve in the DMF, followed by precipitation of the desired product.

The reaction medium is cooled and then filtered through a sinter funnel. The precipitate is washed once with DMF and then thoroughly with methanol.

13.5 g of yellow-white powder are obtained with a yield of 90%.

The mass spectrometry analysis is performed by infusion of the sample in ES+. The product can be dissolved in methanol or toluene by adding TFA.

### Complexation of the Ligand in Organic Medium

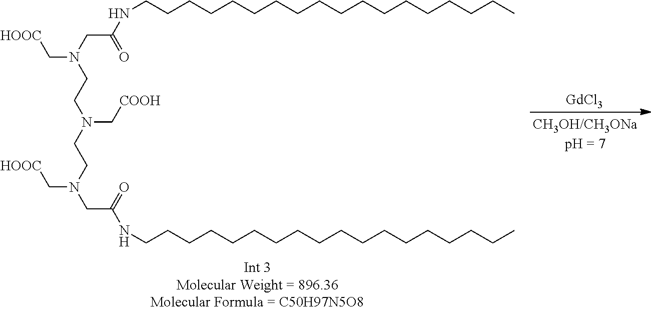 [SMILES: CCCCCCCCCCCCCCCCCCNC(=O)CN(CCN(CCN(CC(=O)O)CC(=O)NCCCCCCCCCCCCCCCCCC)CC(=O)O)CC(=O)O ; "GdCl3"]

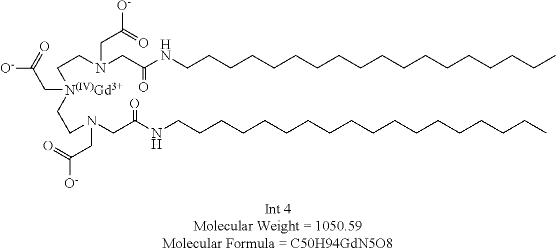

| | M (g/mol) | m (g) | n (mmol) | eq | V (mL) |
|---|---|---|---|---|---|
| Ligand Int 3 | 896.36 | 13.4 | 15 | 1 | |
| GdCl3, 6H2O | 371 | 6.67 | 18 | 1.2 | |
| MeONa/MeOH | | 2.68 | | | 400 |
| MeOH | | | | | 600 |

The ligand (Int3) is suspended in the methanol. The GdCl₃,6H₂O is added. Dissolution takes place instantaneously. The pH of the solution is adjusted to 7 with a solution of sodium methanolate in methanol. The solution is brought to reflux for 45 min. The methanol is evaporated off and the residue is taken up with water. The powder is washed thoroughly with water. 15 g of crude product are obtained with a yield of 96%.

The mass spectrometry analysis is performed by infusion of the sample in ES+. The product can be dissolved in methanol with dichloromethane.

### Purification:

The product is purified by flash chromatography on silica gel. 15 g are purified with an eluent phase composed of 90/10 methanol/dichloromethane.

After purification, 10 g of pure product are obtained (greasy white powder). Purification yield=67%

### EXAMPLE 5

### Synthesis of an Emulsion of PFOB with the Diblock Fluorophilic Compound F6H10 and the Amphiphilic Targeting Ligand of Example 3

1.18 g of egg yolk phosphatidylcholine (EPC) (Lipoid), 220 mg of DSPE-PEG 2000 (Lipoid) and 110 mg of the compound of example 3 are dispersed, with magnetic stirring, in 30 ml of water containing 2.5% w/w of glycerol, for 2 hours, after having been passed through an ultrasonic bath for 20 minutes.

After adjustment to physiological pH, 740 mg of diblock F6H10 are added. Finally, 20 g of PFOB are slowly introduced dropwise as a preemulsifier using an Ultraturrax. The preemulsion is finished with a microfluidizer and then filtered through 0.45 μm.

This composition corresponds to a 40% (w/w) PFOB emulsion. The surfactant composition is 11% (w/w) of surfactants relative to the PFOB or else 16 mmol of surfactant per 100 g of PFOB. The molar proportions in the surfactants are the following: 50% of F6H10 and 50% of non-fluorinated surfactants. The molar composition of non-fluorinated surfactants is 93% of EPC, 5% of DSPE-PEG 2000 and 2% of the compound of example 3.

The emulsion obtained is characterized by a hydrodynamic diameter of 196 nm measured using a nanosizer ZS from Malvern.

### EXAMPLE 6

### Synthesis of an Emulsion of PFOB with the Diblock Fluorophilic Compound F6H10 and the Amphiphilic Targeting Ligand of Example 3 with a Composition Different than that of Example 5

The synthesis protocol is identical to example 5, but using the following composition:

### 20 g of PFOB

### 590 mg of EPC

### 110 mg of DSPE-PEG 2000

28 mg of compound of example 3
370 mg of diblock F6H10
30 ml of aqueous phase.

The PFOB and the diblock F6H10 were purified beforehand according to the procedure described in M. Le Blanc et al., Pharmaceutical research, 246-248 (1985).

This composition corresponds to a 40% (w/w) PFOB emulsion. The surfactant composition is 5.5% (w/w) of surfactants relative to the PFOB or else 8 mmol of surfactant per 100 g of PFOB. The molar proportions in the surfactants are the following: 50% of F6H10 and 50% of non-fluorinated surfactants. The molar composition of non-fluorinated surfactants is: 94% of EPC, 5% of DSPE-PEG 2000 and 1% of the compound of example 3.

The emulsion obtained is characterized by a hydrodynamic diameter of 225 nm measured using a Nanosizer ZS from Malvern.

### EXAMPLE 7

### Synthesis of an Emulsion of PFOB with the Diblock Fluorophilic Compound F6H10 and the Amphiphilic Targeting Ligand of Example 1

The synthesis protocol is identical to example 5, but using the compound of example 1 in place of that of example 3.

### 5 g of PFOB

### 145 mg of EPC

### 30 mg of DSPE-PEG 2000

15 mg of compound of example 1
92 mg of diblock F6H10
20 ml of aqueous phase.

This composition corresponds to a 20% (w/w) PFOB emulsion. The surfactant composition is 5.5% (w/w) of surfactants relative to the PFOB or else 8 mmol of surfactant per 100 g of PFOB. The molar proportions in the surfactants are the following: 50% of F6H10 and 50% of non-fluorinated surfactants. The molar composition of non-fluorinated surfactants is: 93% of EPC, 5% of DSPE-PEG 2000 and 2% of the compound of example 1.

The emulsion obtained is characterized by a hydrodynamic diameter of 178 nm measured using a Nanosizer ZS from Malvern.

### EXAMPLE 8

### Synthesis of an Emulsion of PFOB with the Diblock Fluorophilic Compound F6H10 and the Amphiphilic Targeting Ligand of Example 1 with a Low % of Surfactants

The protocol is identical to example 5, but incorporating the following amounts of surfactants:

### 5 g of PFOB

### 75 mg of EPC

### 15 mg of DSPE-PEG 2000

7 mg of compound of example 1
46 mg of diblock F6H10
20 ml of aqueous phase.

This composition corresponds to a 20% (w/w) PFOB emulsion. The surfactant composition is 2.75% (w/w) of surfactants relative to the PFOB or else 4 mmol of surfactant per 100 g of PFOB. This composition is not in the range [3%-15% by weight]. The molar proportions in the surfactants are the following: 50% of F6H10 and 50% of non-fluorinated surfactants. The molar composition of non-fluorinated surfactants is: 93% of EPC, 5% of DSPE-PEG 2000 and 2% of the compound of example 3.

The emulsion obtained is characterized by a hydrodynamic diameter of 260 nm measured using a Nanosizer ZS from Malvern

### EXAMPLE 9

### Synthesis of an Emulsion of PFOB with the Diblock Fluorophilic Compound F6H10 and the Amphiphilic Targeting Ligand of Example 1 with a Solvent Step

The composition is identical to example 7, but the procedure includes an organic solvent:

EPC, DSPE-PEG 2000, the compound of example 1 and the diblock F6H10 are dissolved in a chloroform/methanol mixture (7/3). The organic phase is evaporated off in a rotary evaporator. The firm obtained is taken up in the aqueous phase and then PFOB is added to the solution. This solution is passed through the Uttraturrax, then through the microfluidizer and finally filtered through 0.45 μm.

The emulsion obtained is characterized by a hydrodynamic diameter of 336 nm.

### EXAMPLE 10

### Synthesis of a PFOB Emulsion with Addition of Rhodamine

The protocol is identical to that of example 5, but incorporating the following amounts of surfactants:

### 20 g of PFOB

### 580 mg of EPC

### 110 mg of DSPE-PEG 2000

55 mg of compound from example 3
370 mg of diblock F6H10

### 2 mg of DSPE-rhodamine (Lipoid)

30 ml of aqueous phase.

This composition corresponds to a 40% (w/w) PFOB emulsion. The surfactant composition is 5.5% (w/w) of surfactants relative to the PFOB or else 8 mmol of surfactant per 100 g of PFOB. The molar proportions in the surfactants are the following: 50% of F6H10 and 50% of non-fluorinated surfactants. The molar composition of non-fluorinated surfactants is: 92.8% of EPC, 5% of DSPE-PEG 2000, 2% of the compound of example 3 and 0.2% of DSPE-rhodamine.

The emulsion obtained is characterized by a hydrodynamic diameter of 198 nm measured using a Nanosizer ZS from Malvern

### EXAMPLE 11

### Synthesis of a PFOB Emulsion with Rhodamine and Gd Complex

The protocol is identical to that of example 5, with the following amounts of surfactants:

### 20 g of PFOB

### 460 mg of EPC

### 110 mg of DSPE-PEG 2000

370 mg of diblock F6H10

### 2 mg of DSPE-rhodamine (Lipoid)

168 mg of compound of example 4
30 ml of aqueous phase.

This composition corresponds to a 40% (w/w) PFOB emulsion. The surfactant composition is 5.5% (w/w) of surfactants relative to the PFOB or else 8 mmol of surfactant per 100 g of PFOB. The molar proportions in the surfactants are the following: 50% of F6H10 and 50% of non-fluorinated surfactants. The composition of non-fluorinated surfactants is: 74.8% of EPC, 5% of DSPE-PEG 2000, 0.2% of DSPE-rhodamine and 20% of Gd complex.

The emulsion obtained is characterized by a hydrodynamic diameter of 210 nm measured using the Nanosizer ZS from Malvern.

### EXAMPLE 12

### Synthesis of an Emulsion of PFOB, without Diblock, with the Specific Ligand of Example 1 (Comparative Example)

The protocol is identical to that of example 5, but the diblock F6H10 is not added. The various compounds are introduced in the following amounts:

### 5 g of PFOB

### 285 mg of EPC

### 15 mg of DSPE-PEG 2000

7 mg of compound of example 1
20 ml of aqueous phase.

This composition corresponds to a 20% (w/w) PFOB emulsion. The surfactant composition is 6% (w/w) of surfactants relative to the PFOB or else 7 mmol of surfactant per 100 g of PFOB. The molar proportions in the surfactants are the following: 98% of EPC, 1.5% of DSPE-PEG 2000 and 0.5% of the compound of example 1.

The emulsion obtained is characterized by a hydrodynamic diameter of 134 nm measured using the Nanosizer ZS from Malvern.

### EXAMPLE 13

### Synthesis of a Crown Ether Emulsion

The protocol and the composition are identical to example 5, with the PFOB being replaced with perfluoro-15-crown-5-ether.

### EXAMPLE 14

### Monitoring in Terms of Stability of the Emulsions of Example 5, 7, 8, 9 and 12

The following table reports the measurements of hydrodynamic diameter measured using the Nanosizer ZS from Malvern at t=0, 3, 9 and 12 months.

| | D_{H} (nm) | D_{H} (nm) | D_{H} (nm) 9 | D_{H} (nm) |
|---|---|---|---|---|
| Product | T0 | 3 months | months | 12 months |
| Example 5 | 196 | 240 | 257 | ND |
| Example 7 | 178 | 230 | 263 | 283 |
| Example 8 | 260 | 313 | 400 | 430 |
| Example 12 | 134 | 202 | 300 | >1 μm |
| Example 9 | 336 | 641 | 300 and 2000 | ND |

The emulsions of example 5, 7 and 8 exhibit good stability at 12 months despite a slight increase in size.

### EXAMPLE 14

### Measurement of the IC50 of the Emulsions of Examples 5, 7, 8, 9 and 12

The measurement of the IC50 of the emulsions is carried out 3 via measurements of competition with αvβ3 on HUVEC cells overexpressing echistatin-¹²⁵I.

The HUVEC suspension is dispensed into a 96-well conical-bottom plate in a proportion of 2×10⁵ cells in 50 μl in binding buffer. Fifty μl of the solutions of increasing concentration of echistatin or RGD products are added per well. The positive control is performed by adding binding buffer without competitor. All the concentration points are carried out in duplicate. The plate is incubated for 2 h at ambient temperature with shaking. Fifty μl of the solution of echistatin-¹²⁵I-SIB at 3 nM are then dispensed into each well and the plate is again incubated for 2 h at ambient temperature with shaking. The reaction mixtures are transferred into vials containing 200 μl of a density cushion composed of paraffin and dibutyl phthalate (10/90). The microtubes are then centrifuged at 12 000 rpm for 3 min. The tubes are finally frozen in liquid nitrogen, and then sectioned in order to count the cell pellet and the supernatant in a gamma counter. A competition curve is then plotted, where the relative binding of the echistatin-¹²⁵I-SIB is determined by the following equation:

$$Relative binding of echistatin \text{-} {I}^{125} \text{-} SIB = \frac{Radioactivity bound in the presence of competitor ( cpm )}{Radioactivity of the control sample ( cpm )} \times 100$$

The data are analyzed using the GraphPad Prism® 5.0 software which determines the IC₅₀ values for each product from the competition curve.

| Compound | IC50 (nM of emulsion) |
|---|---|
| Example 5 | 0.002 |
| Example 7 | 4 |
| Example 8 | 6 |
| Example 9 | 0.7 |
| Example 12 | 75 |

1. An oil-in-water nanoemulsion composition comprising:
an aqueous phase,
a fluorinated phase comprising at least one fluorinated oil,
a surfactant at the interface between the aqueous and fluorinated phases, the surfactant comprising:
at least one amphiphilic targeting ligand, the targeting ligand of which is chosen from: pharmacophores, peptides, pseudopeptides, peptidomimetics, amino acids, peptides and pseudopeptides, integrin-targeting peptidomimetics, glycoproteins, lectins, biotin, pteroic or aminopteroic derivatives, folic acid, folic and antifolic acid derivatives, antibodies or antibody fragments, avidin, steroids, oligonucleotides, ribonucleic acid sequences, deoxyribonucleic acid sequences, hormones, proteins which are optionally recombinant or mutated, mono- or polysaccharides, agents for targeting cell receptors or enzymes,
at least one amphiphilic lipid, and
at least one diblock or tribock fluorophilic compound of formula R_{F}-L-R_{H}(—Z)_{z}
in which:
1) R_{F} is a fluorinated or perfluorinated group (which optionally comprises side chains and/or rings and/or heteroatoms, in particular halogens);
2) R_{H} is a hydrocarbon-based group which optionally comprises side chains and/or rings and/or heteroatoms, in particular halogens and/or multiple bonds;
3) L is a linker group and may comprise in particular one of the following groups: single bond, —CH₂—, —CH═CH—, —O—, —S—, —PO₄—, CONH;
4) Z is H or a group which is more polar or polarizable than the R_{H} groups;
5) z represents 0 or 1.

2. The composition as claimed in claim 1, wherein the diblock or triblock fluorophilic compound is chosen from:
compounds of formula CₙF₂ₙ₊₁CₘH₂ₘ₊₁ (saturated), or of formula CₙF₂ₙ₊₁CₘH₂ₘ₋₁ (unsaturated), or combinations thereof, n being an integer from 2 to 12 and m being an integer from 2 to 16,
compounds of formula CₚH₂ₚ₊₁—CₙF₂ₙ—CₘH₂ₘ₊₁, with p=1-12, m=1-12 and n=2-12,
compounds of formula CₙF₂ₙ₊₁—CH═CH—CₘH₂ₘ₊₁, with n and m, which may be identical or different, between 2 and 12,
substituted ether or polyether compounds of formula XCₙF₂ₙOCₘH₂ₘX, XCF₂OCₙH₂ₙOCF₂X, with n and m=1-4, X=Br, Cl or I,
ether diblock or triblock compounds for formula:
CₙF₂ₙ₊₁—O—CₘH₂ₘ₊₁, with n=2-10; m=2-16, or a)
CₚH₂ₚ₊₁—O—CnF₂ₙ—O—CₘH₂ₘ₊₁, with p=2-12, m=1-12 and n=2-12, b)
where, in each formula, n, m and p are integers.

3. The composition as claimed in claim 1, comprising:
an aqueous phase, preferably representing 29.4% to 80% by weight of the composition, advantageously 55% to 65%, more advantageously from 58% to 62%,
a fluorinated phase comprising at least one fluorinated oil, representing 19.4% to 70% by weight of the composition, advantageously 35% to 45%, more advantageously 37% to 42%,
a surfactant at the interface between the aqueous and fluorinated phases, the surfactant comprising at least one diblock or triblock fluorophilic compound, at least one amphiphilic lipid and at least one amphiphilic targeting ligand,
the total surfactant content by weight relative to the fluorinated oil being between 3% and 10%, advantageously between 4% and 8%,
the total surfactant content by weight relative to the composition being between 0.6% and 7%, advantageously between 1% and 3%.

4. The composition as claimed in claim 1, wherein the surfactant comprises:
non-fluorinated amphiphilic compounds, of which 80 mol % to 95 mol % of amphiphilic lipid, 0 mol % to 5 mol % of pegylated lipids and 0.1 mol % to 10 mol % of amphiphilic targeting ligand,
diblock or triblock fluorophilic compounds;
the non-fluorinated amphiphilic compounds representing 30 mol % to 60 mol % of the total surfactants, and the diblock or triblock fluorophilic compounds representing 30 mol % to 60 mol % of the total surfactants;
advantageously, the non-fluorinated amphiphilic compounds representing 50 mol % of the total surfactants, and the diblock or triblock fluorophilic compounds representing 50 mol % of the total surfactants.

5. The composition as claimed in claim 1, wherein:
1) the surfactant comprises:
50 mol % of diblock or triblock fluorophilic compounds,
50 mol % of non-fluorinated amophiphilic compounds,
2) the 50% of non-fluorinated amphiphilic compounds comprise:
50 mol % to 95 mol % of amphiphilic lipids,
0 to 25 mol % of amphiphilic paramagnetic metal chelate,
0.1 mol % to 10 mol % of amphiphilic targeting ligand,
0 to 10 mol % of pegylated lipids,
0.1 mol % to 0.5 mol % of amphiphilic compounds comprising a fluorophore.

6. The composition as claimed in claim 1, wherein the fluorinated oil is chosen from perfluorocarbons which are linear or branched, or cyclic or polycyclic, and saturated or unsaturated, perfluorinated cyclic tertiary amines, perfluoro esters or thioesters, haloperfluorocarbons; and advantageously: perfluorooctylbromide PFOB, C₈F₁₇Br (PFOB or perfluorobron), perfluorooctylethane (C₈F₁₇C₂H₅ PFOE), perfluorodecalin FDC, perfluorooctane C₈F₁₈, perfluorodichlorooctane, perfluoro-n-octyl bromide, perfluoroheptane, perfluorodecane C₁₀F₂₂, perfluorododecyl bromide C₁₀F₂₂Br PFDB, perfluorocyclohexane, perfluoromorpholine, perfluorotripropylamine, perfluorotributylamine, perfluorodimethylcyclohexane, perfluorotrimethylcyclohexane, perfluorodicyclohexyl ester, perfluoro-n-butyltetrahydrofuran.

7. The composition as claimed in claim 1, wherein the targeting ligand of the amphiphilic targeting ligand is a naphthyridine compound.

8. The composition as claimed in claim 1, wherein the amphiphilic targeting ligand is written in the form:
Bio-L₁-L₂-Lipo
in which:
Bio is a targeting ligand,
Lipo is a lipophilic group for inserting the targeting ligand into the surfactant layer;
L₂ is a linker group, advantageously chosen from:
C₁₋₆ alkylene, PEG, for example CH₂—(CH₂—O—CH₂)ₖ—CH₂ with k=2 to 10, (CH₂)₃—NH, NH—(CH₂)₂—NH, NH—(CH₂)₃—NH, nothing or a single bond, (CH₂)ₙ, (CH₂)ₙ—CO—, —(CH₂)ₙNH—CO— with n being an integer from 2 to 10, (CH₂CH₂O)_{q}(CH₂)ᵣ—CO—, (CH₂CH₂O)_{q}(CH₂)ᵣ—NH—CO— with q being an integer from 1 to 10 and r an integer from 2 to 10, (CH₂)ₙ—CONH—, (CH₂)ₙ—CONH-PEG, (CH₂)ₙ—NH—HOOC—CH₂—O—(CH₂)₂—O—(CH₂)₂—O—CH₂—COOH; HOOC—(CH₂)₂—CO₂—(CH₂)₂—OCO—(CH₂)₂—COOH; HOOC—CH(OH)—CH(OH)—COOH; HOOC—(CH₂)ₙ—COOH; NH₂—(CH₂)ₙ—NH₂, with n being an integer from 0 to 20; NH₂—(CH₂)ₙ—CO₂H; NH₂—CH₂—(CH₂—O—CH₂)ₙ—CO₂H with n being an integer from 1 to 10, squarate
P1-I-P2, P1 and P2, which may be identical or different, being chosen from O, S, NH, nothing (single bond), CO₂, NHCO, CONH, NHCONH, NHCSNH, SO₂NH—, NHSO₂—, squarate,
with I=alkylene, alkoxyalkylene, polyalkoxyalkylene (in particular PEG), alkylene interrupted with one or more squarates or with one or more aryls, advantageously phenyl, alkenylene, alkynylene, alkylene interrupted with one or more groups chosen from —NH—, —O—, —CO—, —NH(CO)—, —(CO)NH—, —O(CO)—, or —(OC)O—,
L₁ is chosen from a single bond, —CONH—, —COO—, —NHCO—, —OCO—, —NH—CS—NH—, —C—S—, —N—NH—CO—, —CO—NH—N—, —CH₂—NH—, —N—CH₂—, —N—CS—N—, —CO—CH₂—S—, —N—CO—CH₂—S—, —N—CO—CH₂—CH₂—S—, —CH═NH—NH—, —NH—NH═CH—, —CH═N—O—, —O—N═CH—, or corresponds to the following formulae:

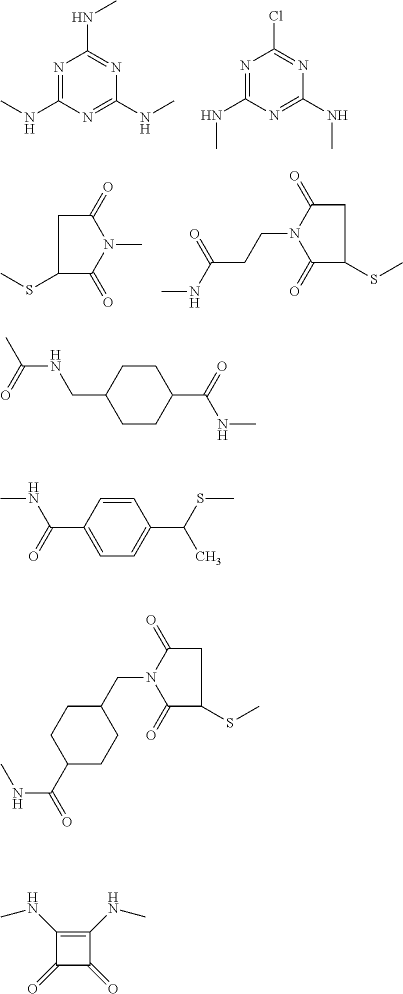

9. A contrast agent comprising a composition as claimed in claim 1.
